(19) United States
(12) Patent Application Publication
(10) Pub. No.: US 2002/0061582 A1
(43) Pub. Date: May 23, 2002

(54) Method of stimulating the mating of microorganisms in liquid medium

(76) Inventors: Michael Albers, Dossenheim (DE); Manfred Kogl, Eppelheim (DE); Ingo Kober, Gaiberg (DE); Eva Loser, Heidelberg (DE)

Correspondence Address: ARENT FOX KINTNER PLOTKIN & KAHN, PLLC, Suite 400, 1050 Connecticut Avenue, N.W., Washington, DC, 20036-5339, US

(21) Appl. No.: 09/963,663

(22) Filed: Sep. 27, 2001

(30) Foreign Application Priority Data
Sep. 27, 2000 (DE) 100 47 951.0-41

(51) Int. Cl. C12N 1/18

(52) U.S. Cl. 435/254.2; 435/254.21

(57) ABSTRACT

Liquid culture media for improved mating efficiency of microorganisms with a sexual or parasexual life cycle are described along with methods for improving mating efficiency of yeast cells in yeast two hybrid systems. The improved mating efficiency of cells is observed with liquid culture media containing high-molecular weight, swellable compounds including polyethylene glycol.

### BACKGROUND OF THE INVENTION

The present invention relates to a method of stimulating the mating of microorganisms with sexual or parasexual life cycle, preferably yeast, e.g. Saccharomyces cerevisiae, in a liquid medium. The method is characterized in that the microorganisms of differing mating types are cultured in the liquid medium in the presence of a high-molecular weight, swellable compound, preferably polyethylene glycol. The present invention also relates to a screening method based on the “yeast two hybrid” system (Y2H).

In the “yeast two hybrid” system which is used, inter alia, in the field of screening for desired substances, two different plasmids are placed jointly in a yeast cell to test whether the two fusion proteins expressed by these plasmids bind to each other. Binding of the two fusion proteins results in the activation of reporter genes, which leads to a measurable phenotypic modification of the yeast cells. As a result of this modification, the cells exhibit histidine-independent growth; cells can grow on agar plates in the absence of histidine. The yeast two hybrid system relates to testing a target protein (catch protein) for interaction with a large number of proteins from a gene library. For this purpose, a yeast strain which expresses the target protein, is contacted with different clones from the gene library. This can be accomplished in different ways, namely by

(a) transforming the yeast strain expressing the target protein with the DNA of the gene library; or

(b) mating yeast cells. Yeast cells are first transformed with a gene library, and those yeast cells carrying the different clones from the gene library express the corresponding proteins. If a haploid yeast culture which expresses the target protein is mixed with a haploid culture carrying the gene library, the yeast cells mate and fuse to form diploid cells. The proviso is that the two strains are each of a different mating type.

However, when gene libraries are analyzed, it is important to be able to test the largest possible number of clones and to identify any rare clones in any given library. This is even more important when many different libraries are tested with a large number of targets. The inventive method overcomes many of these problems associated with art-recognized methods.

Transformation of yeast cells requires several incubations in different media and plating the yeast cells on selective agar plates. This type of method does not lend itself to screening on a relatively large scale. A critical factor in yeast screening methods is mating efficiency. The larger the percentage of cells which fuse to form cygotes, the more material that can be screened. Since only a limited number of yeast cells can be spread over a selective agar plate, large screenings often require many hundred plates. This places a limitation on the number of plates that can be handled in any one screening process. This same limitation applies to other selection methods, such as cell-sorting by fluorescence (FACS), cell-sorting by binding to magnetic particles (MAGS) or selection in liquid media.

Various methods for increasing the amount of diploid cells in the mating process are known in the art. Even the most efficient methods include a step in which the yeast cells are contacted on a solid support such as filter paper or an agar plate. Following incubation, the yeast are washed off the support and further processed. Although these methods achieve a basically good efficiency, they have the disadvantage in that the cells must first be applied to the support and thereafter removed. This process cannot be automated, which further contributes to the inefficiency of the method. Although a method in which the cells are mated in liquid medium is much simpler, the mating efficiency is very low, and this does not lend itself to screening on a large scale.

The present invention overcomes the disadvantages of the prior art methods. An object of the inventive method is to increase the mating efficiency of microorganisms which is simple and cost-effective. This technical problem is solved by the embodiments herein described.

### DETAILED DESCRIPTION OF THE INVENTION

The Inventors have made the surprising discovery that microorganisms can be mated with high efficiency in liquid medium when the medium contains a high-molecular weight, swellable compound. The inventive method is well suited for efficient, high throughput screening since it allows one of ordinary skill in the art to obtain efficient mating of the cells simply by mixing two yeast strains in liquid medium. To achieve enhanced matting efficiency, a high-molecular weight, swellable compound is added to the liquid medium, wherein polyethylene glycol (PEG) is a preferred embodiment. PEG causes aggregation of cells which resembles the state in which the cells are typically found when grown on an agar plate or a filter. Cell aggregation is considered to contribute to increased mating efficiency.

The inventive method also eliminates the use of agar plates and the steps of plating and flushing cells. Thus, the overall material costs that are otherwise associated with art-recognized methods are significantly reduced. Additionally, the inventive method lends itself to industrial scale levels of production, which has not been appreciated much less realized by the prior art methods.

The present invention relates to a method for stimulating the mating of microorganisms with a sexual or parasexual life cycle in a liquid medium, characterized in that microorganisms of different mating type are cultured in the liquid medium in the presence of a high-molecular weight, swellable compound.

This high-molecular weight, swellable compound causes microorganisms to come into physical contact followed by flocculation or agglutination. Preferably, the growth properties of the microorganisms are not negatively affected.

The liquid medium or basic medium for culturing microorganisms, preferably yeast cells, includes but is not limited to YPD, YPAD, SD, SCC or SMM (Adams et al., Methods in Yeast Genetics, Cold Spring Habour Laboratory Press, 1997, ISBN 0-87969-508-0). Other culture conditions, e.g. temperature, O₂ supply, CO₂ supply, etc., correspond to conditions routinely used for culturing the respective microorganisms, and these culture conditions are well known to one skilled in the art.

All microorganisms having a sexual or parasexual life cycle are suitable for the inventive, and include but are not limited to Escherichia coli or Bacillus subtilis.

In a preferred embodiment, the microorganisms are yeasts including klyveromyces, hansenula, saccharomyces, or schizosaccharomyces, more preferably Saccharomyces cerevisiae, Schizosaccharomyes pombe, Klyveromyces lactis or Hansenula polymorpha, and most preferably, Saccharomyces cerevisiae or Schizosaccharomyces pombe.

The high-molecular weight, swellable compound is a compound which dehydrates the medium and leads to microorganisms coming into contact followed by flocculation or agglutination of microorganisms in liquid media. These compounds include but are not limited to agarose beads, Sepharose® beads, cellulose powder, Sephadex®, PEG, etc. The term “high-molecular weight” used herein, relates to a molecular weight range of 1,000 or more, preferably, 1,000 to 150,000 daltons, more preferably 3,000 to 100,000 daltons, and most preferably 5,000 to 50,000 daltons.

In a preferred embodiment of the inventive method, PEG is added to the liquid medium, preferably in the form of a solid and having a molecular weight over 400 daltons. More preferably, the polyethylene glycol has a molecular weight ranging from 2,000 to 20,000, and most preferably, a molecular weight of 6,000 daltons.

The concentration of the compound for increasing the mating efficiency in the liquid medium is not critical, and a person skilled in the art can determine the optimum concentration (i.e., the concentration resulting in the maximum mating efficiency without essentially impairing the growth or the survival rate of the cells) by the method described herein below. The optimum concentration also depends on the respective compound and the respective microorganism, and the combination thereof. In a preferred embodiment, the compound is present in the liquid medium at a concentration of 2 to 12 % by weight, and a concentration of 10 % by weight being most preferred.

The optical density (OD₆₀₀) of the microorganism suitable for maximum mating efficiency in liquid medium, and the duration of incubation are not critical and can also be determined by the person skilled in the art by means of simple experiments (e.g., by the method described below). The optimum optical density (OD₆₀₀) and the duration of incubation also depend on the respective compound and the respective microorganism, and the combination thereof. In a preferred embodiment, microorganisms are incubated in liquid medium with an OD₆₀₀ between 1.0 and 4.0, preferably for 3 to 4 hours.

A more preferred embodiment of the inventive method comprises the steps of

(a) providing a first microorganism of one mating type and comprising a gene encoding a reporter protein and a second microorganism of another mating type transformed with a gene library,

(b) co-incubating the first and second microorganism to form a yeast two hybrid cell and to activate expression of the reporter protein; and

(c) selecting the yeast two hybrid cell of step (b) expressing the reporter protein.

The person skilled in the art is familiar with methods of isolating microorganisms from liquid media, e.g. centrifugation. Methods of constructing gene libraries or recombinant vectors for expressing a reporter protein (or target protein) are also known to the person skilled in the art. Suitable vectors, preferably expression vectors, are also known to the person skilled in the art. The vectors may be a plasmid or another suitable vehicle for gene expression. The vector is preferably functionally linked to regulatory elements permitting its expression in eukaryotic host cells. Preferred plasmids and vectors are pAD-GAL4-2.1, pBD-GAL4, pBD-GAL4Cam, pCMV-AD, PCMV-BD, pMyr, psos, pACT2, pAS2-1, pHISi, pLexA, pM, pHISi-1, pB42AD, pVP16, pGAD10, pGBKT7, pLacZi, p8op-lacZ, PGAD GH, pGilda, pAD GL, pGADT7, pGBDU, pDBLeu, pPC86, or pDBTrp. Such vectors contain in addition to the regulatory elements, a promoter, typically a replication origin, and specific genes which permit the phenotypic selection of a transformed host cell. The regulatory elements for the expression in yeast cells include the AOX1 or GAL1 promoter, ADH promoter, GAL1-10 promoter, Cup promoter or Met1 promoter. Vectors suitable for the expression in yeast are pY100 or Ycpad1 as well as the above-listed vectors, in particular those of the pRS series, the YCP series or the YEP series (Guthrie, C. and Fink, G. (editor), 1991, Methods in Enzymology 194, Guide to Yeast Genetics and Molecular Biology, Academic Press, San Diego, Calif. 92101, ISBN 0-12-182095-5). Methods for the transformation of microorganisms and the phenotypic selection of transformants are known in the art or described sufficiently in the literature; see e.g. Sambrook et al., Molecular Cloning: A Laboratory Manual, 2nd edition, Cold Spring Harbor Laboratory Press, Cold Spring Harbor, N.Y. (1989).

The person skilled in the art is also familiar with suitable reporter proteins or target proteins. Reporter proteins serve to function in the screening of molecules from the gene library. In a preferred embodiment, a yeast two hybrid cell would contain a molecule donated by a cell containing a cloned gene from the library and a gene encoding the reporter protein donated by the second cell. Under appropriate conditions, the cloned molecule would activate the expression of the reporter gene to express the reporter protein, and the reporter protein could be used in selection of the inducing molecule obtained from the library (Fields S, Song O., A novel genetic system to detect protein-protein interactions, Nature 1989, 340 (6230); 245-246; Walhout AJ, Vidal M., Protein interaction maps for model organisms, Nat. Rev. Mol. Biol. 2001, 2(1): 55-62). Reporter proteins include but are not limited to green, yellow or red fluorescent protein, β-galactosidase, α-galactosidase, β-lactamase, chloramphenical-acetyl transferase, β-glucuronidase, ADE2, ADE3, Leu2, His3, peroxidase, alkaline phosphatase, luciferase, or surface antigens.

The following example explains the invention.

### EXAMPLE

### Mating of yeast cells in liquid medium with 10% PEG 6000

Deep-frozen yeast cells (strain CG-1945 or AH109, Matalpha, Clontech yeast protocol handbook PT3024-1, Clontech company, Palo Alto, U.S.A.) of a Y2H library from human brain (order number HL4004AH, 1 ml; Clontech company, Palo Alto, U.S.A.) were thawed and incubated at 30° C. for 1 hour in YPDA medium (10 g Bacto yeast extract, 20 g Bacto peptones, 20 g glucose, 40 mg adenine sulfate, water ad 1000 ml; sterilizing by autoclaving; ingredients from the company of DIFCO Laboratories, Detroit, U.S.A.) . Cells of opposite mating type (MATa) which expressed the ligand binding domain of the human PPARgamma protein in fusion with the DNA binding domain of the pGBKT7 vector (Clontech company, Palo Alto, U.S.A., catalog # K1612-B) (cell with target or reporter protein) were grown overnight in selective medium to an OD₆₀₀ of 1.0. Twenty O.D. units of cells of both yeast strains were mixed with one another, centrifuged off and resuspended in 15 ml YPDA medium. The OD₆₀₀ was measured as 3.75. 7.5 ml of these mixtures each, were distributed between two Erlenmeyer flasks. 1.9 ml of a 50 % solution of PEG 6000 (company of Carl Roth, Karlsruhe, Germany) was added to one of the two flasks, so that the final concentration of PEG 6000 was about 10 %. Both cultures were incubated at 30° C. while shaking (100 rpm) for four hours. Thereafter, the cells were centrifuged off, washed once in selective medium without leucine, histidine and tryptophan (Adams et al., Methods in Yeast Genetics, Cold Spring Habour Laboratory Press, 1997, ISBN 0-87969-508-0) and resuspended in 30 ml of the same medium. The cells were diluted to a concentration of 1:500 in the same medium, and 50 μl were plated onto different plates (see Table 1). Following incubation at 30° C. for 3 days, the number of colonies was determined. The results are shown in Table 1.

TABLE 1

| | Without PEG 6000 | 10% PEG 6000 |
|---|---|---|
| leucine (cells | 132 | 215 |
| with the gene | | |
| library) | | |
| tryptophan (cells | about 1000 | about 1000 |
| with the target | | |
| protein) | | |
| leucine, | 9 | 149 |
| tryptophan | | |
| (diploid cells) | | |

Table 1 shows that, following the correction to the same number of surviving cells from the library (plates-leucine), the quantity of diploid cells existing when 10% PEG is present on plates without leucine and tryptophane (=selection for diploids) is ten times as much as that without PEG. Thus, about 22 to 35 million diploids were formed in the 15 ml medium of the mating experiments carried out here when PEG was used for stimulating the mating, whereas only 1.35 million diploids were present without PEG.

What is claimed:

1. A method for stimulating mating of microorganisms with a sexual or parasexual life cycle in a liquid medium, comprising incubating microorganisms of two different mating types in the liquid medium comprising a high-molecular weight, swellable compound.

2. The method according to claim 1, wherein the microorganisms are yeast.

3. The method according to claim 2, wherein the yeast is Saccharomyces cerevisiae or Schizosaccharomyces pombe.

4. The method according to claim 1, wherein the high-molecular weight, swellable compound is polyethylene glycol.

5. The method according to claim 4, wherein polyethylene glycol has a molecular weight ranging from 2,000 to 20,000 daltons.

6. The method according to claim 5, wherein polyethylene glycol has a molecular weight of about 6,000 daltons.

7. The method according to claim 1, wherein the high-molecular weight, swellable compound is present in a concentration ranging from 2 to 12 % by weight of the liquid medium.

8. The method according to claim 7, wherein the concentration is 10% by weight.

9. The method according to claim 1, wherein the microorganisms in the liquid medium are incubated at an OD₆₀₀ between 1.0 and 4.0.

10. An method for improving mating efficiency of yeast cells in a yeast two hybrid system comprising incubating the yeast cells in a liquid culture medium containing a high-molecular weight, swellable compound.

11. An aqueous culture system for yeast cell mating comprising a liquid culture medium and a high-molecular weight, swellable compound.

12. An improved yeast two hybrid system for library screening comprising
(a) providing a first microorganism of one mating type comprising a gene encoding a reporter protein and a second microorganism of another mating type transformed with a gene library,
(b) co-incubating the first and second microorganism in a liquid medium comprising a high-molecular weight, swellable compound to obtain a yeast two hybrid and for activating expression of the reporter protein in the yeast two hybrid; and
(c) screening the library by selecting the yeast two hybrid of step (b) expressing the reporter protein.
